(19) United States
(12) Patent Application Publication
(10) Pub. No.: US 2017/0081300 A1
(43) Pub. Date: Mar. 23, 2017

(54) SYDNONE DERIVATIVES FOR CONJUGATION OF COMPOUNDS OF INTEREST

(71) Applicants: UNIVERSITE DE STRASBOURG, STRASBOURG (FR); CENTRE NATIONAL DE LA RECHERCHE SCIENTIFIQUE, PARIS (FR); COMMISSARIAT A L'ENERGIE ATOMIQUE ET AUX ENERGIES ALTERNATIVES, PARIS (FR)

(72) Inventors: FREDERIC TARAN, GIF-SUR-YVETTE (FR); ALAIN WAGNER, STRASBOURG (FR); SERGII KOLODYCH, ORSAY (FR); OLEKSANDR KONIEV, STRASBOURG (FR)

(21) Appl. No.: 15/310,459

(22) PCT Filed: May 15, 2015
(86) PCT No.: PCT/EP2015/060805

§ 371 Date: Nov. 11, 2016

(30) Foreign Application Priority Data
May 16, 2014 (EP) 14305726.3

(51) Int. Cl. C07D 271/04; C07D 495/04; C07D 231/54; C07K 16/00; C07K 14/765

(52) U.S. Cl. CPC C07D 271/04; C07K 16/00; C07K 14/765; C07D 231/54; C07D 495/04

(57) ABSTRACT

The present invention relates to a process for the preparation of a first compound of interest C1 functionalized with a sydnone compound and to the corresponding functionalized C1 compound of interest. The present invention also relates to a process for the preparation of a conjugate of two compounds of interest C1 and C2 implying a sydnone compound and to the obtained conjugate. The present invention also relates to a process for preparing a compound of interest C2 comprising a strained alkyne moiety functionalized with a sydnone and to the corresponding functionalized compound of interest C2. It also relates to novel sydnone compounds substituted in position 4, which may be used in the above processes.

### INTRODUCTION

The present invention relates to a process for the preparation of a first compound of interest C1 functionalized with a sydnone compound and to the corresponding functionalized C1 compound of interest. The present invention also relates to a process for the preparation of a conjugate of two compounds of interest C1 and C2 implying a sydnone compound and to the obtained conjugate. The present invention also relates to a process for preparing a compound of interest C2 comprising a strained alkyne moiety functionalized with a sydnone and to the corresponding functionalized compound of interest C2. It also relates to novel sydnone compounds substituted in position 4, which may be used in the above processes.

### BACKGROUND OF THE INVENTION

The present invention relates to the domain of click chemistry (bioorthogonal reaction) and of bioconjugation of compounds of interest. Five main approaches are currently available for metal-free coupling two different compounds of interest ( and ▪). These approaches are presented on the scheme below.

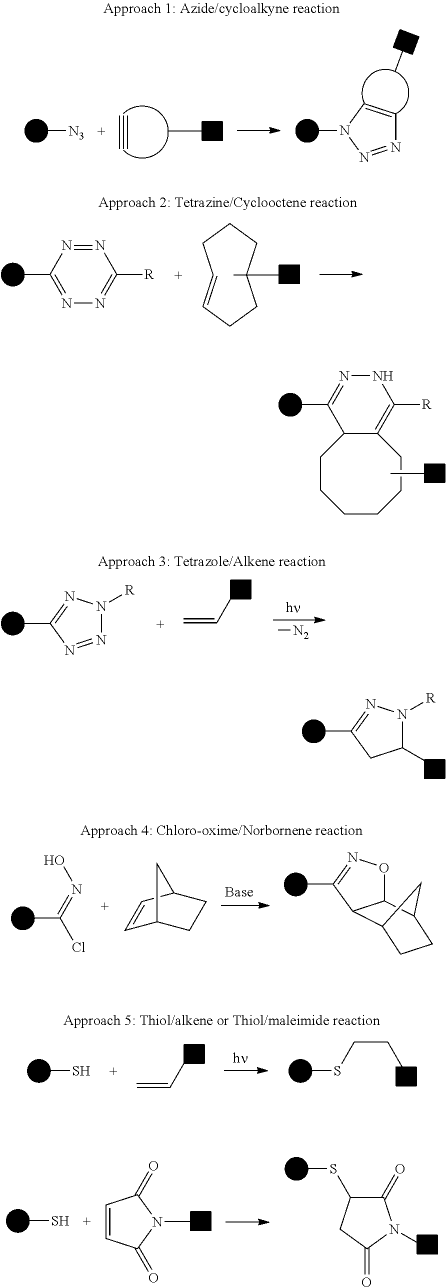

Bioconjugation reactions, for instance bioconjugation reactions involving proteins, are often performed in highly dilute conditions, for instance because of the low available amount of the starting biomaterial, or because the solubility of said starting biomaterial is low in the coupling conditions.

Consequently, it is important in the domain of bioorthogonal reactions to use efficient coupling reagents allowing high yields and fast coupling kinetics.

The inventors of the present application recently discovered that reaction of a sydnone and an alkyne could be efficiently used to perform efficient bioorthogonal reactions (Kolodych et al. Angew. Chem. Int. Ed. 2013, 52, 12056-12060).

Browne et al. (J. Org. Chem. 2010, 75, 984-987) disclose the cycloaddition of 4-iodosydnones with terminal alkynes. The cycloaddition reactions disclosed by Browne et al. are performed at elevated temperatures (140° C. or 200° C.) in a non polar solvent (xylenes). Such conditions are not appropriate for any domain of application; for instance, such conditions are not compatible with reactions involving biomolecules, such as bioconjugation reactions. In addition, Browne et al. further disclose that this cycloaddition reaction of iodosydnones may not be transposed to bromosydnones because bromosydnones would be unstable at elevated temperatures.

Wallace et al. (Chem. Sci. 2014, published 5 Feb. 2014) recently described the cycloaddition of phenylsydnone, which is not substituted in position 4, on a bicyclononyne.

The applicant of the present invention surprisingly evidenced that the use of a specific family of sydnone derivatives, which are substituted in position 4 with specific groups, affords a great increase in efficiency and kinetics of the coupling reactions. These sydnone compounds can be advantageously used for the conjugation of compounds of interest. They also found that some of these sydnone compounds had not been described so far and were thus novel.

### SUMMARY OF THE INVENTION

The present invention relates to a process for preparing a compound, of formula (III):

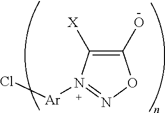

wherein n is an integer from 1 to 100,
comprising the step of contacting a compound of interest C1 with a sydnone of formula (I):

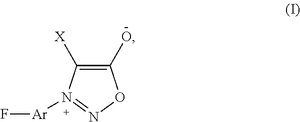

wherein
X is a halogen atom selected from the group consisting of chlorine, bromine and fluorine atoms, an aryldiazo group, an alkyl group, a phenyl group substituted with at least one electrodonating group, such as an alkoxyl group or a dialkylamino group, an alkenyl group, an alkynyl group an alkoxyl group or an amino group;
Ar is an aromatic group; and
F is selected from specific functional groups, namely:
a carboxylic acid COOH group,
a thiol SH group,
a maleimide

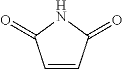

group,
an activated ester, such as a N-hydroxysuccinimide, a N-hydrophthalimide ester, a perfluorinated ester or an acylurea,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group as defined above, and
an alkyl group substituted by at least one of the groups above.

Another object of the invention is a functionalized compound of interest C1 of formula (III), which may be obtained according to the above process.

Another object of the invention is a process for preparing a conjugate, comprising a step of contacting a compound of interest C2 comprising a strained alkyne moiety of formula (IV)

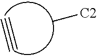

with a functionalized compound of interest C1 of formula (III) according to the invention.

Another object of the invention is a conjugate of formula (II):

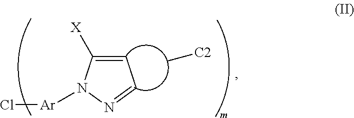

wherein C1 is a first compound of interest, C2 is a second compound of interest and m is an integer from 1 to 100.

Another object of the invention is a process for preparing a functionalized compound, C2 of formula (V):

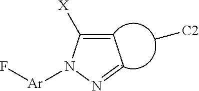

comprising the step of contacting a compound of formula (IV):

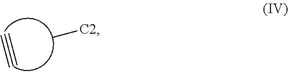

wherein C2 is a compound of interest, with a sydnone of formula (I) as described above.

Another object of the invention is a functionalized compound of interest C2 of formula (V), which may be obtained by the process described above.

Another object of the invention is a process for preparing a conjugate, comprising a step of contacting a compound of interest C1 with a functionalized compound of interest C2 of formula (V) according to the invention.

Still another object of this invention is a novel class of sydnones of formula (I′):

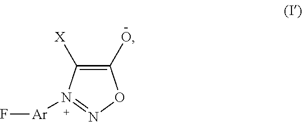

wherein
X is a halogen atom selected from the group consisting of chlorine, bromine and fluorine atoms, an aryldiazo group, an alkyl group, a phenyl group substituted with at least one electrodonating group, an alkenyl group, an alkynyl group, an alkoxyl group or an amino group,
Ar is an aromatic group selected from the group consisting of:
   a phenyl group;
   a heteroaromatic group; and
   a polyaromatic group;
   wherein the phenyl, heteroaromatic and/or polyaromatic group may be optionally substituted with at least one substituent selected from the group consisting of a halogen atom, an alkyl group, an alkoxyl group, a carboxyl COOH group and a nitro NO₂ group, and
F is a functional group allowing bonding to a compound of interest C1, wherein the functional group F is selected from the group consisting of:
   a carboxylic acid COOH group,
   a thiol SH group,
   a maleimide

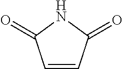

group,
an activated ester, such as a N-hydroxysuccinimide, a N-hydrophthalimide ester, a perfluorinated ester or an acylurea,
a iodine atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group as defined above, and
an alkyl group substituted by at least one of the groups above,
   wherein, if Ar is an optionally substituted phenyl group, F is in para position of the phenyl group.

### BRIEF DESCRIPTION OF THE FIGURES

FIG. 1: HPLC monitoring of the kinetics of the cycloaddition reaction involving a chlorosydnone in blood plasma.

FIG. 2: ¹H NMR spectroscopic analysis of the competitive reaction between a chlorosydnone and an azide with the cyclooctyne BCN in CD₃OD at 25° C.

FIG. 3: Linear regressions for the kinetics of the reaction of two sydnones of the invention with BCN (bicyclononyne).

FIG. 4: Comparison of protein labeling efficiencies with a chlorosydnone versus the corresponding non-chlorinated sydnone.

### DETAILED DESCRIPTION OF THE INVENTION

A first object of the invention is a sydnone compound of formula (I′):

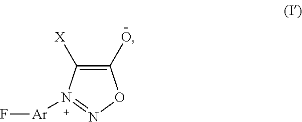

wherein:
X is a halogen atom selected from the group consisting of chlorine, bromine and fluorine atoms, an aryldiazo group, an alkyl group, a phenyl group substituted with at least one electrodonating group, an alkenyl group, an alkynyl group, an alkoxyl group or an amino group,
Ar is an aromatic group selected from the group consisting of:
   a phenyl group;
   a heteroaromatic group; and
   a polyaromatic group;
   wherein the phenyl, heteroaromatic and/or polyaromatic group may be optionally substituted with at least one substituent selected from the group consisting of a halogen atom, an alkyl group, an alkoxyl group, a carboxyl COOH group and a nitro NO₂ group, and
F is a functional group selected from the group consisting of:
   a carboxylic acid COOH group,
   a thiol SH group,
   a maleimide

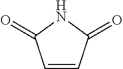

group,
an activated ester, such as a N-hydroxysuccinimide, a N-hydrophthalimide ester, a perfluorinated ester or an acylurea,
a iodine atom,
an alkene or alkyne group, optionally interrupted by at least one heteroatom selected among O, N and S,
   an amino (—NRR′) group, wherein R and R′ are independently hydrogen atoms, alkyl, alkene, alkyne or aryl groups as defined above,
   a hydroxylamine (—ONH₂) group,
   a hydrazine (—NH—NH₂) group,
   an azido (—N₃) group,
   a diazonium (—N₂⁺) group, optionally in presence of a counterion,
   a boronic acid —B(OR″)₂ group, wherein R″ is a hydrogen atom or an alkyl group,
   an isocyanate (—N═C═O) or isothiocyanate (—N═C═S) group,
   a chlorosulfonyl (—SO₂Cl) group,
   a —C≡C—C≡N group,
   an aldehyde CHO group,
   a ketone COR′ group, wherein R′″ is an alkyl group as defined above, and
   an alkyl group substituted by at least one of the groups above,
      wherein, if Ar is an optionally substituted phenyl group, F is in para position of the phenyl group.

The counterion can be any ion appropriate for compensating the charge of the diazonium group, and may be easily chosen by anyone of ordinary skill in the art. For instance, the counterion may be selected from the group consisting of halogenates, BF₄⁻, NO₃⁻, HSO₄⁻, PF₆⁻CH₃COO⁻, N(SO₂CF₃)₂⁻, CF₃SO₃⁻, CH₃SO₃⁻, CF₃COO⁻, (CH₃O)(H)PO₂⁻ and N(CN)₂⁻.

An alkyl group is a linear, branched or cyclic saturated hydrocarbon group comprising from 1 to 20 carbon atoms. Preferably, the alkyl group according to the invention comprises from 1 to 10, in particular from 1 to 6, carbon atoms. Examples of alkyl groups comprise methyl, ethyl, propyl, isopropyl, butyl, tertbutyl, isobutyl, n-pentyl, n-hexyl and cyclohexyl groups.

An alkenyl group (or an alkene) is a linear, branched or cyclic hydrocarbon group comprising from 2 to 20 carbon atoms and comprising at least one C═C double bond. Preferably, the alkenyl group according to the invention comprises from 2 to 10, in particular from 2 to 6, carbon atoms. Examples of alkenyl groups comprise ethylenyl, propylenyl and cyclohexenyl groups.

An alkynyl group (or an alkyne) is a linear, branched or cyclic hydrocarbon group comprising from 2 to 20 carbon atoms and comprising at least one C≡C triple bond.

Preferably, the alkynyl group according to the invention comprises from 2 to 10, in particular from 2 to 9, carbon atoms. Examples of alkynyl groups comprise ethynyl, propynyl, octynyl, cyclooctynyl and cyclononynyl groups.

In specific embodiments of the present invention, the alkyl, alkenyl and/or alkynyl groups may be interrupted by at least one heteroatom, preferably selected from nitrogen, oxygen and sulfur atoms.

According to the invention, the alkyl groups, alkenes and alkynes are not substituted by an aryl group.

An alkoxyl group is an alkyl group, bonded to the rest of the molecule through an oxygen atom.

An aryl diazo group is a N₂ group bonded to an aromatic group. A carboxyl group is a COOH group. A nitro group is a NO₂ group.

An amino group is a NR₁R₂ group, wherein R₁ and R₂ are independently selected from the group consisting of hydrogen atoms and alkyl groups. In an embodiment, at least one of R₁ and R₂ is an alkyl group. Such an amino group is an alkylamino group. In an embodiment, R₁ and R₂ are both alkyl groups. Such an amino group is a dialkylamino group.

According to the present invention, an aromatic group (or an aryl) is selected from the group consisting of:
a phenyl group;
a heteroaromatic group such as pyridine, thiophene, imidazole, thiazole, pyrazole, pyrole or furane; and
a polyaromatic group such as anthracene or phenanthrene;
wherein the phenyl, heteroaromatic and/or polyaromatic group may be optionally substituted with at least one substituent (in addition to the F group), preferably selected from the group consisting of a halogen atom, an alkyl group, an alkoxyl group, a carboxyl COOH group and a nitro NO₂ group.

In an embodiment, the aromatic group is an optionally substituted phenyl group, preferably a non-substituted phenyl group. In such a case, the F group is in the para position. When Ar is an optionally substituted heteroaromatic or polyaromatic group, the functional group F can be in any position of the aromatic group.

In a preferred embodiment, the functional group F is selected from the group consisting of:
a carboxylic acid COOH group,
a thiol SH group,
a maleimide

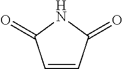

group,
an activated ester, such as a N-hydroxysuccinimide, a N-hydrophthalimide ester, a perfluorinated ester or an acylurea,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂Cl) group,
a —C≡C—C≡N group, and
an alkyl group substituted by at least one of the groups above.

In a highly preferred embodiment, the functional group F is selected from the group consisting of a carboxylic acid COOH group, an activated ester, such as a N-hydroxysuccinimide, a N-hydrophthalimide ester, a perfluorinated ester or an acylurea, and an alkyl group substituted by at least one of these groups. Most preferably, F is a COOH group.

A compound of interest may be for instance a molecule, such as a fluorophore, for instance rhodamine, a group of atoms comprising at least one radioactive atom (¹⁴C, ³H, or ¹³¹I for instance), a group of atoms of known mass (a mass tag), a ligand, a drug, a therapeutic agent, a biomolecule, such as an antibody, a protein, such as BSA (bovine serum albumin), a DNA fragment, a nanoobject, such as a nanoparticle (ie an object or a particle of 0.1 to 1000 nm), or a support, such as a polymer.

In an embodiment, X is selected from the group consisting of a halogen atom selected from the group consisting of chlorine, bromine and fluorine atoms, an aryl diazo group, an alkyl group, a phenyl group substituted with at least one electrodonating group, such as an alkoxyl group or a dialkylamino group, an alkenyl group, an alkynyl group and an amino group.

In a preferred embodiment, X is a halogen atom selected from the group consisting of chlorine, bromine and fluorine atoms, in particular a chlorine or bromine atom.

In another preferred embodiment, X is an alkenyl group, an alkynyl group, or an aryldiazo group.

The sydnones of the invention may be synthesized by any appropriate method known by one of ordinary skill in the art.

For instance, the scheme below presents different synthesis routes for sydnones according to the invention. The Ar groups substituting the nitrogen atom of the sydnones may be substituted with a F group.

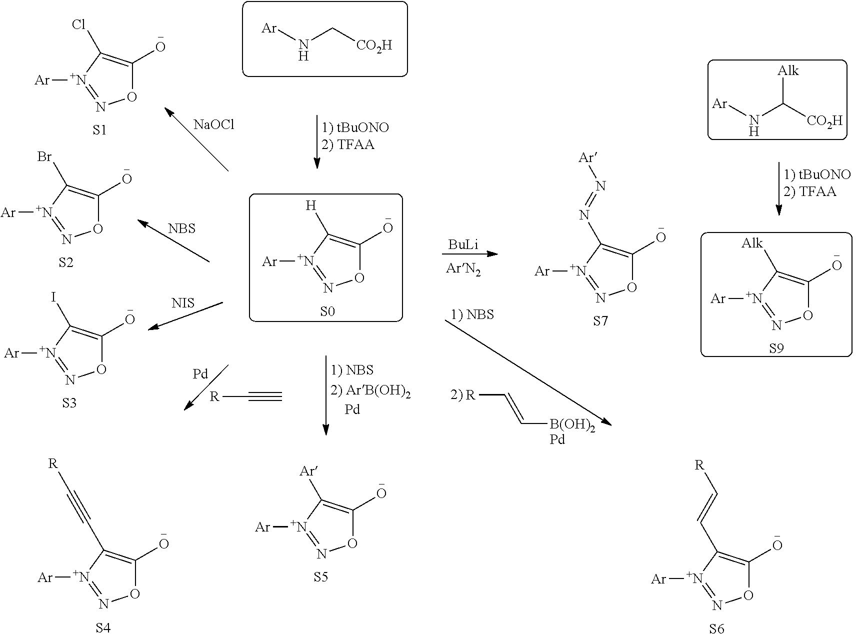

Another object of the invention is a process for the preparation of a functionalized compound of formula (III):

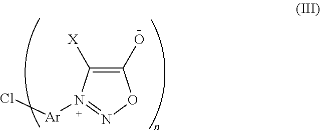

wherein n is an integer from 1 to 100, comprising the step of contacting a compound of interest C1 with a sydnone compound of formula (I)

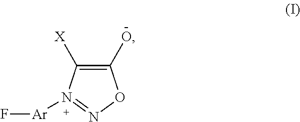

wherein
X is a halogen atom selected from the group consisting of chlorine, bromine and fluorine atoms, an aryldiazo group, an alkyl group, a phenyl group substituted with at least one electrodonating group, an alkenyl group, an alkynyl group, an alkoxyl group or an amino group,
Ar is an aromatic group, and
F is a functional group selected from the group consisting of:
   a carboxylic acid COOH group,
   a thiol SH group,
   a maleimide

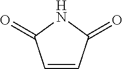

group,
an activated ester, such as a N-hydroxysuccinimide, a N-hydrophthalimide ester, a perfluorinated ester or an acylurea,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′ group, wherein R′″ is an alkyl group as defined above, and
an alkyl group substituted by at least one of the groups above.

A halogen atom is a chlorine, iodine, bromine or fluorine atom. Preferably, a halogen atom is a bromine or a chlorine atom, in particular a chlorine atom.

Another object of the invention is a functionalized compound of interest C1 of formula

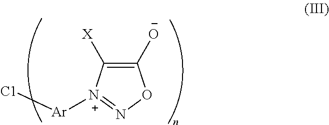

obtainable, preferably obtained, by the above process, wherein n is an integer and preferably ranges from 1 to 100. In an embodiment, n is from 1 to 50, preferably from 1 to 30. In a specific embodiment, n is 1.

In the present invention, the term “functionalized compound” refers to a compound that is bonded to a sydnone compound of formula (I).

The compound of interest C1 is preferably a compound as defined above with at least one group able to covalently bond to a F group, such as
a carboxylic acid COOH group,
a thiol SH group,
a maleimide

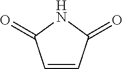

group,
an activated ester, such as a N-hydroxysuccinimide, a N-hydrophthalimide ester, a perfluorinated ester or an acylurea,
a halogen atom,
an alkene or alkyne group, optionally interrupted by at least one heteroatom selected among O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂Cl) group,
a —C≡C—C≡N group,
an aldehyde CHO group,
a ketone COR′″ group, wherein R′″ is an alkyl group as defined above, or
an alkyl group substituted by at least one of the groups above.

In an embodiment, the group able to covalently bond to a F group of C1 is selected from the group consisting of:
a carboxylic acid COOH group,
a thiol SH group,
a maleimide

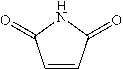

group,
an activated ester, such as a N-hydroxysuccinimide, a N-hydrophthalimide ester, a perfluorinated ester or an acylurea,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂Cl) group,
a —C≡C—C≡N group, and
an alkyl group substituted by at least one of the groups above.

In a preferred embodiment, the group able to covalently bond to a F group of C1 is selected from the group consisting of a carboxylic acid COOH group, an activated ester, such as a N-hydroxysuccinimide, a N-hydrophthalimide ester, a perfluorinated ester or an acylurea, and an alkyl group substituted by at least one of these groups.

If the compound of interest C1 comprises several groups able to covalently bond to F groups, the process may lead to a compound of interest C1 functionalized with several sydnones.

The process of preparation of a functionalized compound of interest C1 according to the invention may comprise a preliminary step of covalent bonding of at least one moiety comprising a reactive group to C1, wherein the reactive group is able to covalently bond to the functional group F of the sydnone compound of formula (I).

Another object of the invention is a process for the preparation of a conjugate of formula (II)

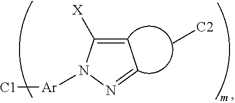

comprising a step of contacting a compound of interest C2 comprising a strained alkyne moiety of formula (IV)

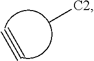

preferably a strained cyclic alkyne moiety, in particular a cyclooctyne moiety, with a functionalized compound of interest C1 of formula (III) according to the invention.

The process for the preparation of a conjugate may comprise a preliminary step of covalent bonding of a moiety comprising a strained alkyne to the compound of interest C2.

According to the present invention, the terms “strained alkyne” refer to an alkyne wherein the triple bond is sterically strained, such as a mono or multi cyclic alkyne comprising from 6 to 12 carbon atoms. For instance, the strained alkyne may be a cyclooctyne, in particular it is bicyclononyne BCN.

The sydnone compounds of formula (I) can be used advantageously in order to couple both compounds of interest C1 and C2.

In an embodiment, C1 is a fluorophore or a group of atoms comprising at least one radioactive atom, and C2 is a biomolecule, a nanoobject or a polymer comprising, or functionalized with, a strained alkyne moiety. In this embodiment, the sydnone affords the labelling of the biomolecule, nanoobject or polymer. In a particular embodiment, C2 is an antibody and this embodiment leads to the formation of a labelled antibody.

In another embodiment, C1 is a nanoobject or a biomolecule, and C2 is a biomolecule, such as an antibody. In this embodiment, the sydnone of formula (I) affords the bioconjugation of the first nanoobject or biomolecule and the second biomolecule.

In another embodiment, C1 is a therapeutic agent, and C2 is a biomolecule, such as an antibody. In this embodiment, the sydnone of formula (I) affords the bioconjugation therapeutic agent and the biomolecule. In a particular embodiment, C2 is an antibody and this embodiment leads to the formation of a therapeutic antibody.

One of the main goals of the present invention is to couple two different compounds of interest C1 and C2. Consequently, the nature of C1 and C2 can be inverted. This means that the invention also encompasses the (C2, C1) couples of compounds of interest even if the examples provided above are for (C1, C2) couples.

The sydnone compounds of formula (I) and the compounds of interest C1 functionalized with a sydnone according to the invention can be coupled very efficiently to strained alkynes, such as cyclic alkynes, in particular cyclooctynes. The corresponding coupling products are obtained with higher yields and faster kinetics than with the current competitive coupling systems. For instance, the applicant has unexpectedly proven that these compounds react up to 30 times faster than the non substituted sydnone disclosed by Wallace on strained cyclic alkynes.

The coupling reaction implies the formation of a cycloadduct formed by a [3+2] cyclization, said cycloadduct then turning to a stable pyrazole by retro-Diels Alder reaction, triggering release of CO₂. The reaction of a sydnone compound according to the invention with a cycloalkyne is represented as example on the scheme below.

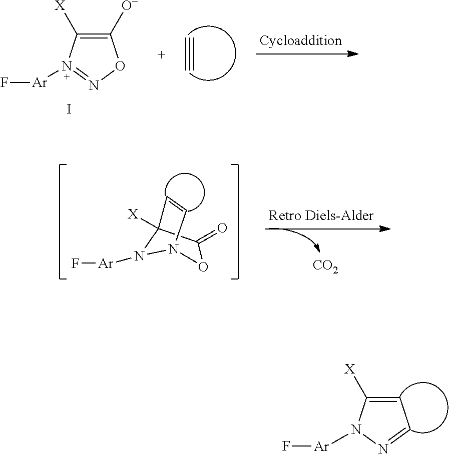

The present compounds allow the efficiency of the reaction to be maintained in biological media, such as culture media, cell lysates or plasma. In addition, as the coupling reaction involving the sydnones of the invention is very rapid, in particular when compared to the other possible coupling reactions, for instance the other coupling reactions used in click-chemistry, the reaction can be efficiently performed even in highly dilute conditions, which is classically the case for instance for bioconjugation reactions. For example, the sydnones of formula (I) allow high level of fluorescent labeling of a protein used at 50 μg/mL in only 5 min, when only a lower amount of labeling could be obtained even after 16 hours with a non-substituted sydnone as disclosed by Wallace (example 4). Taking into account this result, the sydnones would allow coupling reactions on proteins at concentration below 1 μg/mL. The superior activity of these sydnones, such as chlorosydnones, is also illustrated in example 3 and FIG. 2 which correspond to a competitive coupling reaction towards a cyclooctyne with an azide compound (which reacts faster than non-substituted sydnones). Competition is in favor of the chlorosydnone for more than 95%.

Another object of the invention is a conjugate of formula (II):

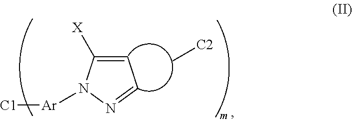

wherein C1 is a first compound of interest, C2 is a second compound of interest and m is an integer and preferably ranges from 1 to 100. m is inferior or equal to n as defined above. In an embodiment, m is equal to n. In a specific embodiment, m is 1.

Another object of the invention is a process for the preparation of a functionalized compound C2 of formula (V):

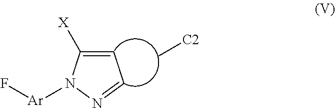

comprising the step of contacting a compound of formula (IV):

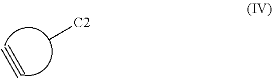

wherein C2 is a compound of interest with a sydnone of formula (I).

The compound of interest C2 is as disclosed above.

This process may comprise a preliminary step of covalent bonding of a strained alkyne moiety to the compound of interest C2.

Another object of the invention is a functionalized compound of interest C2 of formula (V)

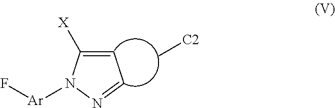

obtainable, preferably obtained, by the above process.

Another object of the invention is a process for the preparation of a conjugate of formula (II)

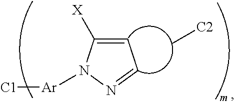

comprising a step of contacting a compound of interest C1 with a functionalized compound of interest C2 of formula (V) according to the invention.

The conjugate of formula (II)

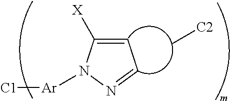

is preferably obtained by one of the processes according to the invention.

The following examples are provided as illustrative, and not limitative, of the present invention.

### EXAMPLES

### Example 1

### Synthesis of Sydnones

In the following examples, the provided yields are molar yields unless specified differently.

### Synthesis of Sydnone S2-2 According to the Invention

### 4-bromo-3-(4-carboxyphenyl)-1,2,3λ⁵-oxadiazol-3-ylium-5-olate

### a) Synthesis of Sydnone S0-4 (3-(4-carboxyphenyl)-1,2,3λ⁵-oxadiazol-3-ylium-5-olate)

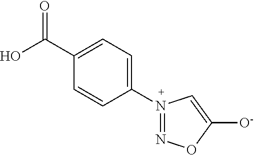

To a vigorously stirred suspension of pCO₂H-phenyl glycine (1.58 g, 8.10 mmol) in 10% aqueous HCl (8.10 mL) at 0° C. was added dropwise, over a period of 40 min, a solution of NaNO₂ (0.56 g, 8.10 mmol) in water (8.10 mL). The resulting mixture was stirred at room temperature under argon for 14 h. The precipitate was collected by filtration, washed with small amount of methanol and dried to obtain 1.54 g (6.90 mmol, 85%) of the intermediate nitroso compound. The later (1.30 g, 5.80 mmol) was stirred at 100° C. for 3 h in acetic anhydride (6.70 mL). The resulting solution was concentrated under vacuum. The residue was triturated with water (10 mL), the precipitate was collected by filtration and recrystallized from methanol. 622 mg (3.00 mmol, 52%) of compound S0-4 were isolated as a white solid.

¹H NMR (400 MHz, DMF-d₇, δ ppm): 8.32 (d, J=8.5 Hz, 2H), 8.19 (d, J=8.5 Hz, 2H), 7.88 (s, 1H). ¹³C NMR (100 MHz, DMF-d₇, δ ppm): 169.7, 167.1, 138.9, 135.7, 132.3, 123.1, 96.1. LCMS (ESI) m/z: 207 [M+H]⁺. Mp.: 232-234° C.

### 12) Synthesis of Sydnone S2-2

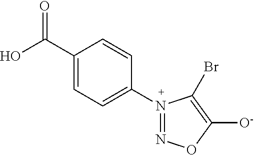

N-Bromosuccinimide (1.1 eq) was added to a stirred solution of sydnone S0-4 (0.50 mmol, 1 eq) in 1.2 mL of acetic acid. The mixture was allowed to stir at room temperature for 2 h after which time 5 mL of water was added. The resultant precipitate was isolated by filtration to afford the brominated compound as an orange solid (44%).

¹H NMR (400 MHz, Acetone-d₆, δ ppm): 8.39 (d, J=8.6 Hz, 2H), 8.03 (d, J=8.6 Hz, 2H). ¹³C NMR (100 MHz, Acetone-d₆, δ ppm): 166.1, 166.0, 138.3, 135.3, 132.1, 126.7, 85.9. IR (KBr, cm⁻¹): 2820, 2087, 1942, 1904, 1850, 1743, 1606, 1591, 1509, 1445, 1423, 1335, 1317, 1290, 1205, 1127, 1114, 1035, 1018, 974, 942, 881, 858, 820, 808. LCMS (ESI): m/z: 285 [M(⁷⁹Br)+H]⁺, 287 [M(⁸¹Br)+H]⁺. Mp.: 117-119° C. (decomp.)

### Synthesis of Sydnone S1-2 According to the Invention

### 3-(4-carboxyphenyl)-4-chloro-1,2,3λ⁵-oxadiazol-3-ylium-5-olate

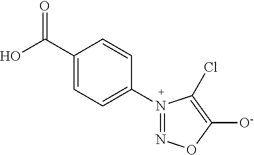

A solution of sodium hypochlorite 10% (1.23 mL, 2 mmol) was added dropwise to a stirred solution of carboxyphenylsydnone (206 mg, 1.00 mmol) in a dioxane/HCl 1M 2/1 mixture (12 mL). The mixture was allowed to stir at room temperature for 4 h. The mixture was poured in a solution of Na₂S₂O₃ 20%. EtOAc was added and the aqueous layer was extracted 3 times with EtOAc. The organic layers were combined, dried over MgSO₄ and concentrated under reduced pressure. The crude product was purified by silica gel column to afford the chlorinated compound as an orange solid (40%).

¹H NMR (400 MHz, Acetone-d₆, δ ppm): 8.30 (d, J=8.6 Hz, 2H), 7.90 (d, J=8.6 Hz, 2H). IR (KBr, cm⁻¹): 3315, 2927, 2549, 1791, 747, 1691, 1607, 1510, 1455, 1425, 1346, 1320, 1290, 1219, 1176, 1128, 1113, 1043, 1018, 182, 972, 945, 886, 859, 808. LCMS (ESI): m/z: 241 [M(³⁵C1)+H]⁺, 243 [M(³⁷C1)+H]⁺.

### Synthesis of 4-bromo-3-(4-nitrophenyl)-1,2,3λ⁵-oxadiazol-3-ylium-5-olate (Comparative Sydnone)

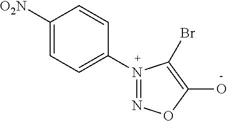

N-Bromosuccinimide (97.9 mg, 0.55 mmol) was added to a stirred solution of nitrophenylsydnone (104 mg, 0.50 mmol) in 1.2 mL of acetic acid. The mixture was allowed to stir at room temperature for 2 h after which time 5 mL of water was added. The resultant precipitate was isolated by filtration to afford 4-bromo-3-(4-nitrophenyl)-1,2,3λ⁵-oxadiazol-3-ylium-5-olate (70%).

¹H NMR (400 MHz, Acetone-d₆, δ ppm): 8.39 (d, J=8.6 Hz, 2H), 8.03 (d, J=8.6 Hz, 2H). ¹³C NMR (100 MHz, Acetone-d₆, δ ppm): 166.1, 166.0, 138.3, 135.3, 132.1, 126.7, 85.9. IR (KBr, cm⁻¹): 3112, 3091, 3066, 1945, 1937, 1907, 1855, 1755, 1614, 1594, 1527, 1496, 1449, 1374, 1344, 1315, 1294, 1204, 1164, 1105, 1030, 1013, 195, 966, 885, 861, 848. LCMS (ESI): m/z: 286 [M(⁷⁹Br)+H]⁺, 288 [M(⁸¹Br)+H]⁺. Mp.: 150-152° C.

### Synthesis of 3-(4-nitrophenyl)-1,2,3λ⁵-oxadiazol-3-ylium-5-olate (Comparative Sydnone)

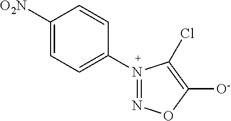

N-Chlorosuccinimide (134 mg, 1.00 mmol) was added to a stirred solution of nitrophenylsydnone (104 mg, 0.50 mmol) in 1.2 mL of acetic acid. The mixture was allowed to stir at room temperature for 6 h. The crude product was purified by silica gel column chromatography (eluent: Dichloromethane/MeOH 99/1, AcOH 1%) to afford 3-(4-nitrophenyl)-1,2,3λ⁵-oxadiazol-3-ylium-5-olate.

¹H NMR (400 MHz, CDCl₃, δ ppm): 8.29 (m, 2H), 8.14 (m, 2H). IR (KBr, cm⁻¹): 3315, 3123, 3054, 2639, 2500, 1935, 1773, 1721, 1659, 1610, 1595, 1551, 1513, 1493, 1418, 1409, 1389, 1376, 1341, 1328, 1302, 1275, 1244, 1203, 1175, 1111, 981, 855, 814.

### Example 2

### Kinetic Study of the Reaction of Sydnones According to the Invention with Cyclic Alkynes

Reactions of sydnone S1-2 with bicyclononyne BCN was carried out in blood plasma at 100 μM concentration of sydnone and 150 μM concentration of cyclooctyne BCN using the following procedure:

To 900 μL of blood plasma were added 10 μL of the solution of benzamide (internal standard, 100 mM in DMSO), 1 μL of the solution of sydnone (100 mM in DMSO) and 1.5 μL of the solution of BCN (100 mM in DMSO). The reaction mixture was injected in HPLC every 30 min and the conversion was followed by measuring the normalized sydnone peak area.

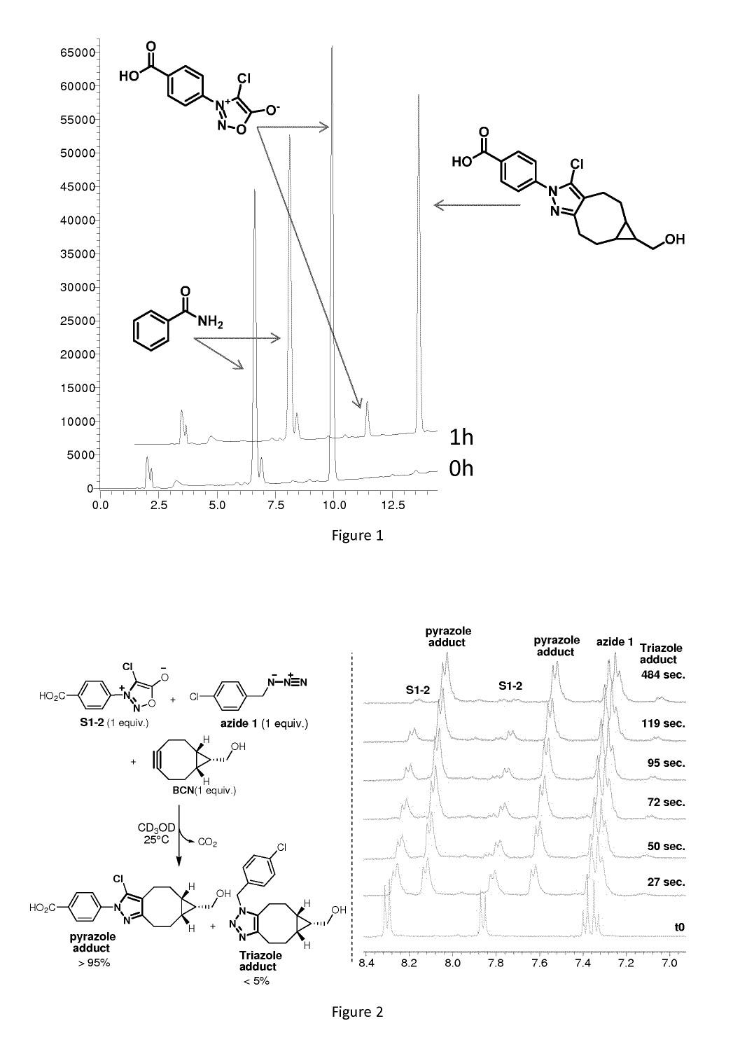

FIG. 1 presents the HPLC monitoring of the reaction between sydnone S1-2 of the invention and the cyclic alkyne BCN. The reaction has been conducted at 100 μM in blood plasma. The results indicated an almost complete reaction after 1 hour in blood plasma, proving that the reaction is fast enough to be useful to bioconjugation application in complex biological media.

### Example 3

### Comparison with the Coupling with Azides

0.1 mmol of sydnone S1-2, azide 1

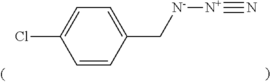 [SMILES: N#[N+][N-]Cc1ccc(Cl)cc1]

and BCN were dissolved in CD₃OD at room temperature. The reaction was followed by NMR on the aromatic part of the spectrum which provides specific NMR signals.

FIG. 2 presents the evolution of the NMR spectrum in function of time of a competitive cycloaddition reaction between the sydnone of the invention S1-2 and an azide with the cyclooctyne BCN. The results clearly confirmed the superior activity of the chlorosydnone over azide to undergo reaction with BCN.

### Example 4

### Kinetic Study of the Reaction of Different Sydnones with Cyclic Alkynes

The kinetics of the coupling reaction of different sydnones with two cyclic alkynes was studied, according to the following scheme.

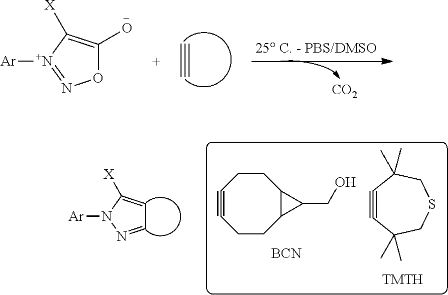

Reactions of sydnones with BCN (or TMTH) were carried out in PBS/DMSO (9:1) mixtures at 100 μM concentration of sydnones and 150 μM concentration of BCN using the following procedure:

To 900 μL of phosphate buffered saline (PBS, 100 mM) was added 87.5 μL of DMSO, 10 μL of the solution of benzamide (internal standard, 100 mM in DMSO), 1 μL of the solution of sydnone (100 mM in DMSO) and 1.5 μL of the solution of BCN (100 mM in DMSO). The reaction mixture was injected in HPLC every 30 min and the conversion was followed by measuring the normalized sydnone peak area.

Table 1 below presents the kinetic constant values K for these reactions, depending on the Ar group and the X group. Sydnones S1-2 and S2-2 are according to the invention.

TABLE 1

| Syn- | | | | K |
|---|---|---|---|---|
| drome | Ar | X | K (BCN) | (TMTH) |
| S0-1 | pOMeC₆H₄ | H | 0.006 ± 0.001 | 18.4 |
| S0-2 | pMeC₆H₄ | H | 0.032 ± 0.001 | n.d. |
| S0-3 | C₆H₅ | H | 0.027 ± 0.002 | 183 |
| S0-4 | pCO₂HC₆H₄ | H | 0.059 ± 0.001 | n.d. |
| S0-5 | pCF₃C₆H₄ | H | 0.199 ± 0.002 | n.d. |
| S0-6 | pNO₂C₆H₄ | H | 0.289 ± 0.012 | n.d. |
| S9-1 | C₆H₅ | CH₃ | 0.018 ± 0.002 | 16.1 |
| S5-1 | C₆H₅ | C₆H₅ | 0.027 ± 0.001 | n.d. |
| S5-2 | C₆H₅ | pOMeC₆H₄ | 0.015 ± 0.001 | n.d. |
| S6-1 | C₆H₅ | 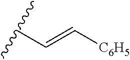 | 0.015 ± 0.001 | n.d. |
| S4-1 | C₆H₅ | 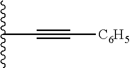 | 0.113 ± 0.001 | n.d. |
| S8-1 | C₆H₅ | SMe | 0.044 ± 0.001 | n.d. |
| S7-1 | C₆H₅ | 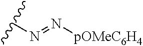 | 0.02 ± 0.001 | n.d. |
| S1-1 | C₆H₅ | Cl | 0.872 ± 0.034 | n.d. |
| S2-1 | C₆H₅ | Br | 0.592 ± 0.021 | n.d. |
| S3-1 | C₆H₅ | I | 0.306 ± 0.008 | n.d. |
| S2-2 | pCO₂HC₆H₄ | Br | 0.798 ± 0.065 | 101 |
| S1-2 | pCO₂HC₆H₄ | Cl | 1.593 ± 0.034 | 322 |

K unit is M⁻¹.sec⁻¹, n.d. = not determined

These results clearly show that the sydnones comprising a halogen atom in position 4 afford a faster reaction than the sydnones comprising no substitution or another substituent in position 4. In particular, 4-chloro-sydnones were found to react with the cyclooctyne BCN more than approximately 30 times faster than sydnones that are non substituted in position 4. Actually, the ratios of the K constants of sydnones according to the invention to the K constants of the corresponding sydnones that are not substituted in position 4 are the following: K(S1-1)/K(S0-3)=32, and K(S1-2)/K(S0-4)=27.

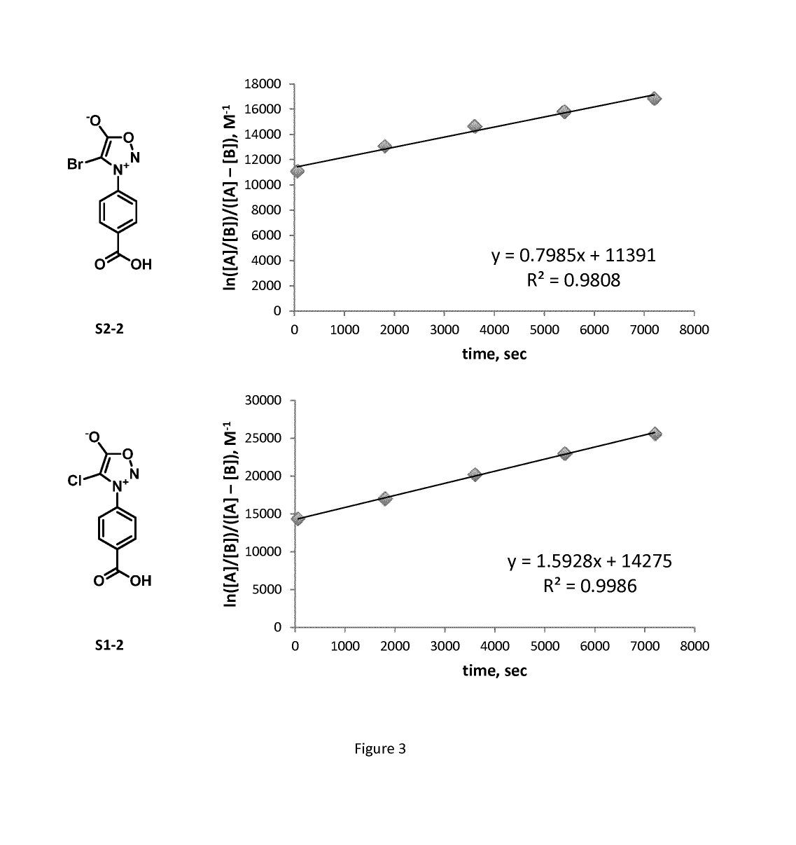

Second order reaction rate was determined by plotting ln([A]/[B])/([A]−[B]) versus time and analyzing by linear regression. Second order rate constant corresponds to the determined slope. Linear regression curves for the sydnones of the invention are presented in FIG. 3.

### Example 4

### Coupling of a Sydnone According to the Invention with a Protein as Compound of Interest C1 and its Use for Fluorescence Labeling with a Rhodamine Derivative as Compound of Interest C2

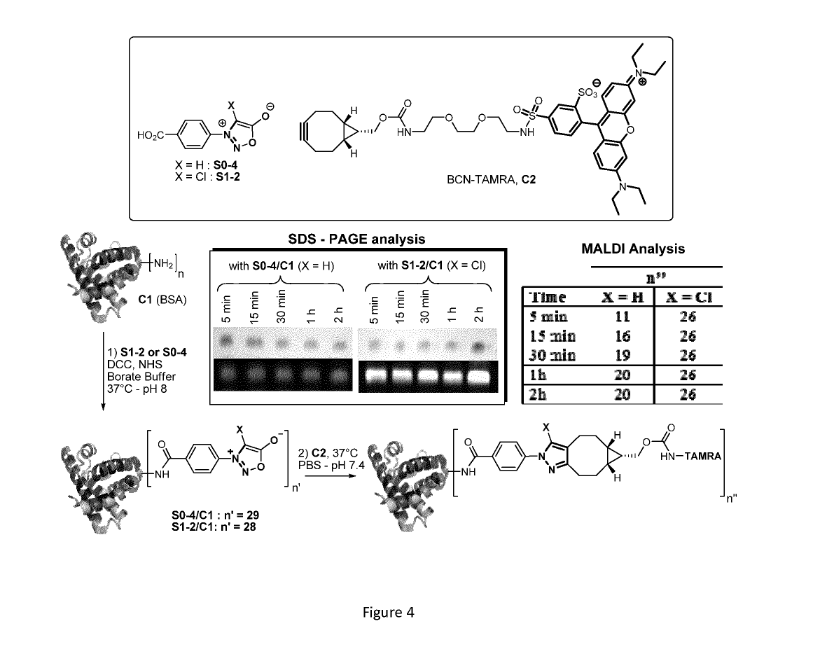

The sydnone S1-2 of the invention was coupled to bovin serum albumin (BSA), used as model protein, by activation of the F function of the sydnone followed by standard peptide coupling to the protein. The corresponding protein-sydnone conjugate was then reacted with a rhodamine (TAMRA)-cyclooctyne conjugate. Sydnone S0-4 which has no substituent in position 4 was also used for comparison. The SDS-PAGE and MALDI results presented in FIG. 4 clearly indicate a better efficiency and a better kinetic of the sydnone of the invention for the rhodamine labeling of the protein.

### Preparation of BSA-Sydnone Conjugates S1-2/C1 and S0-4/C1:

To a solution of N-hydroxysuccinimide (25 μL, 44.64 μmol, 1 eq) in DMF was added a solution of dicyclohexylcarbodiimide (25 μL, 44.64 μmol, 1 eq) in DMF and a solution of sydnone (250 μL, 44.64 μmol) in DMF. The mixture was stand at room temperature during 1 h and was directly used for the conjugation with BSA protein.

A solution of BSA (250 μL, 72 nmol) in phosphate buffer (100 mM, pH 7.4) and 950 μL of borax buffer (0.05 M, pH 9.2) were added to the previous solution. The resulting mixture was incubated during 1 h at 37° C. and purified by size-exclusion chromatography on Sephadex® G-25 medium gel (eluted with 5 mM potassium phosphate buffer pH 8).

29 S0-4 residues and 28 S1-2 residues per BSA protein were conjugated according to MALDI TOF-MS analyses.

### Preparation of BSA-Sydnone-Cycloalkyne-TAMRA Conjugates:

A solution of BCN-POE3-NH-lissamine rhodamine B conjugate (51.2 μL, 130 nmol, 1 eq/sydnones) in MeOH was added to a solution of the BSA-sydnone conjugate (50 μg/mL) in phosphate buffer (5 mM, pH 7.4) and stand at room temperature. Sampling for electrophoresis and MALDI-TOF analysis were performed at 5 min, 15 min, 30 min, 1 h and 16 h. The samples were concentrated with a centrifugal filter unit Amicon® Ultra Millipore™ Ultracel®-10K and frozen to −80° C.

MALDI-TOF-MS analyses were performed on Voyager™ Biospectrometry™ Workstation. SDS-PAGE was performed on PhastGel™ GE Healthcare™ Gradient 10-15 gel using PhastGel™ GE Healthcare™ SDS buffer Strips. Fluorescence was visualized on Molecular Imager® VersaDoc™ MP 4000 system prior to staining with Coomassie Blue.

1-15. (canceled)

16. A sydnone compound of formula (I′) 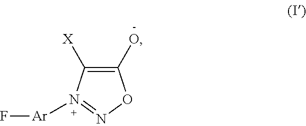
wherein
X is a halogen atom selected from the group consisting of chlorine, bromine and fluorine atoms, an aryldiazo group, an alkyl group, a phenyl group substituted with at least one electrodonating group, an alkenyl group, an alkynyl group, an alkoxyl group or an amino group,
Ar is an aromatic group selected from the group consisting of:
a phenyl group;
a heteroaromatic group; and
a polyaromatic group;
wherein the phenyl, heteroaromatic and/or polyaromatic group may be optionally substituted with at least one substituent selected from the group consisting of a halogen atom, an alkyl group, an alkoxyl group, a carboxyl (COOH) group and a nitro (NO₂) group, and
F is a functional group selected from the group consisting of:
a carboxylic acid (COOH) group,
a thiol (SH) group,
a maleimide 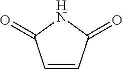
group,
an activated ester a perfluorinated ester or an acylurea,
a iodine atom,
an alkene or alkyne group, optionally interrupted by at least one heteroatom selected from O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂C1) group,
a —C≡C—C≡N group,
an aldehyde CHO group,
a ketone COR′″ group, wherein R′″ is an alkyl group as defined above, and
an alkyl group substituted by at least one of the groups above,
wherein, if Ar is an optionally substituted phenyl group, F is in para position of the phenyl group.

17. The sydnone compound according to claim 16, wherein X is a halogen atom selected from the group consisting of chlorine, bromine and fluorine atoms.

18. The sydnone compound according to claim 16, wherein X is an alkenyl group, an alkynyl group or an aryldiazo group.

19. The sydnone compound according to claim 16, wherein the aromatic group is an optionally substituted phenyl group.

20. The sydnone compound according to claim 16, wherein the functional group F is selected from the group consisting of:
a carboxylic acid (COOH) group,
a thiol (SH) group,
a maleimide 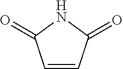
group,
an activated ester a perfluorinated ester or an acylurea,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
an isocyanate (—N═C═O) or isothiocyanate (—N═C═S) group,
a chlorosulfonyl (—SO₂C1) group,
a —C≡C—C≡N group, and
an alkyl group substituted by at least one of the groups above.

21. The sydnone compound according to claim 18, wherein X is a phenyl group substituted with at least one electrodonating group selected from an alkoxyl group and a dialkyamino group.

22. A process for the preparation of a compound of formula (III): 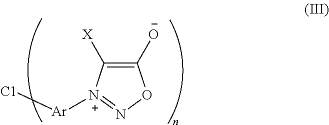
wherein n is an integer from 1 to 100, comprising the step of contacting a compound of interest C1 with a sydnone compound of formula (I) 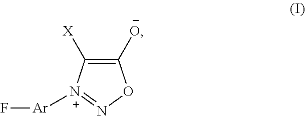
wherein
X is a halogen atom selected from the group consisting of chlorine, bromine and fluorine atoms, an aryldiazo group, an alkyl group, a phenyl group substituted with at least one electrodonating group, an alkenyl group, an alkynyl group, an alkoxyl group or an amino group,
Ar is an aromatic group, and
F is a functional group selected from the group consisting of:
a carboxylic acid (COOH) group,
a thiol (SH) group,
a maleimide 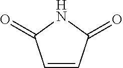
group,
an activated ester, a perfluorinated ester or an acylurea,
a halogen atom,
an alkene or alkyne group, optionally interrupted by at least one heteroatom selected from O, N and S,
an amino (—NRR′) group, wherein R and R′ are independently hydrogen atoms, alkyl, alkene, alkyne or aryl groups as defined above,
a hydroxylamine (—ONH₂) group,
a hydrazine (—NH—NH₂) group,
an azido (—N₃) group,
a diazonium (—N₂⁺) group, optionally in presence of a counterion,
a boronic acid —B(OR″)₂ group, wherein R″ is a hydrogen atom or an alkyl group,
an isocyanate (—N═C═O) or isothiocyanate (—N═C═S) group,
a chlorosulfonyl (—SO₂C1) group,
a —C≡C—C≡N group,
an aldehyde CHO group,
a ketone COR′″ group, wherein R′″ is an alkyl group as defined above, or an alkyl group substituted by at least one of the groups above.

23. A functionalized compound of interest C1 of formula (III) 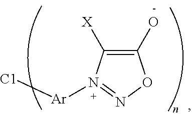
wherein n is an integer from 1 to 100, obtainable by the process according to claim 22.

24. A process for the preparation of a conjugate, comprising a step of contacting a compound of interest C2 comprising a strained alkyne moiety of formula (IV): 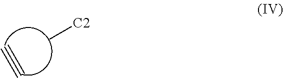
with the functionalized compound of interest C1 of formula (III) according to claim 23.

25. A conjugate of formula (II): 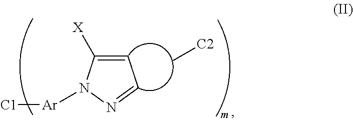
wherein C1 is a first compound of interest, C2 is a second compound of interest and m is an integer from 1 to 100.

26. The conjugate according to claim 25, wherein C1 is a fluorophore and C2 is a biomolecule, or C1 is a therapeutic agent and C2 is a biomolecule.

27. The conjugate according to claim 26, wherein the biomolecule is an antibody.

28. A process for the preparation of a compound of formula (V): 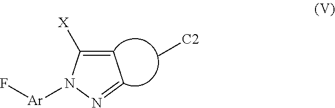
comprising the step of contacting a compound of formula (IV): 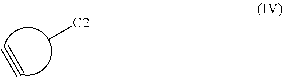
wherein C2 is a compound of interest with a sydnone as defined in claim 22.

29. A functionalized compound of interest C2 of formula (V) 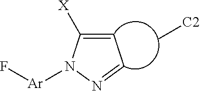
obtainable by the process of preparation according to claim 28.

30. A process for the preparation of a conjugate, comprising a step of contacting a compound of interest C1 with a functionalized compound of interest C2 of formula (V) according to claim 29.
